(19) United States
(12) Patent Application Publication
(10) Pub. No.: US 2019/0133465 A1
(43) Pub. Date: May 9, 2019

(54) DEVICE FOR A NON-INVASIVE BLOOD PRESSURE MEASUREMENT

(71) Applicant: Edwards Lifesciences Corporation, Irvine, CA (US)

(72) Inventors: Ilja Guelen, Ravenstein (NL); Geertruida Lucretia van der Sar, Alphen aan den Rijn (NL); Jeroen Van Goudoever, Amstelveen (NL); Peiyuan Li, Amsterdam (NL); Jacobus Jozef Gerardus Maria Settels, De Hoef (NL)

(21) Appl. No.: 16/174,740

(22) Filed: Oct. 30, 2018

Related U.S. Application Data
(60) Provisional application No. 62/581,228, filed on Nov. 3, 2017.

(51) Int. Cl. A61B 5/0225; A61B 5/022; A61B 5/0235; A61B 5/00; A61B 5/0295

(52) U.S. Cl. CPC A61B 5/02255; A61B 5/02241; A61B 5/0235; A61B 2562/0233; A61B 5/0295; A61B 2562/0247; A61B 5/742

(57) ABSTRACT

A device for a non-invasive pressure blood pressure measurement, comprising: a pressure cuff, for placement around a body part such as a finger; which cuff comprises: a bladder, for wrapping around the body part; a light source, for sending light through the body part; and a light detector, for detecting the light passed through the body part and for providing a signal in dependence of the amount of detected light; a first fluid reservoir, in fluid connection with the bladder, to supply fluid to the bladder; a second fluid reservoir, in fluid connection with the bladder, to receive fluid from the bladder; a pressure generator, for generating a differential pressure between the first and second fluid reservoir; a variable flow resistance, located between the fluid reservoirs and the bladder, and a controller, arranged to control the variable flow resistance, and to determine the blood pressure inside the body part.

### CROSS-REFERENCE TO RELATED APPLICATIONS

This application claims priority to U.S. Provisional Patent Application No. 62/581,228, filed Nov. 3, 2017, the contents of which are incorporated herein by reference.

### TECHNICAL FIELD

The present invention relates to a finger cuff for a non-invasive blood pressure measurement.

### BACKGROUND OF THE INVENTION

It has been known for several years to measure blood pressures wherein a pressure cuff is placed around a body extremity, such as a finger. EP 0 048 060 for instance describes that the pressure of a fluid inside the pressure cuff is controlled on the basis on a signal of a plethysmograph by a pressure valve, in turn controlled by a control loop.

In the known systems, ambient air is pressurized and supplied to the pressure cuff to increase the pressure inside the pressure cuff. To decrease the pressure inside the cuff, air inside the cuff is released to the ambient again. Although these systems function in practise, they are relatively large and require large volumes of air to be pumped from and to the ambient.

It is therefore an objective of the present invention to provide an improved device for a non-invasive pressure blood pressure measurement.

### SUMMARY

The invention provides a device for a non-invasive blood pressure measurement, comprising: a pressure cuff, for placement around a body part such as a finger; which cuff comprises: a bladder, for applying a pressure to the body part; a volume determining unit for providing a signal based on the volume of blood flowing through the body part, for example a light source, for sending light through the body part and a light detector, for detecting the light passed through the body part and for providing the signal in dependence of the amount of detected light; a first fluid reservoir, in fluid connection with the bladder, to supply fluid to the bladder; a second fluid reservoir, in fluid connection with the bladder, to receive fluid from the bladder; a pressure generator, for generating a differential pressure between the first and second fluid reservoir; a variable flow resistance, located between the fluid reservoirs and the bladder, and a controller, arranged to control the variable flow resistance based on the signal. The controller may further be arranged to determine the blood pressure inside the body part.

Instead of pressurizing and releasing ambient air, the device according to the invention proposed to utilise a substantially closed system. The pressure generator generates a differential pressure between two fluid reservoirs, such as air tanks. Compared to ambient pressure, one fluid reservoir is at a positive pressure, while the other reservoir is at a negative pressure. The two reservoirs are connected to the inflatable bladder of the pressure cuff through a variable flow resistance, such as a valve or a flapper. The two reservoirs may also be connected to the inflatable bladder, each through their own variable flow resistance.

The variable flow resistance for instance opens connection of the bladder with the positive pressure reservoir when fluid is needed to be supplied to the bladder. When pressure in the bladder is to be reduced, the variable flow resistance may close the connection with the positive pressure reservoir, and open connection of the bladder with the negative pressure reservoir. Due to the pressure difference between the bladder and the (negative) pressure reservoir, fluid is actively sucked out of the bladder, and into the negative pressure reservoir. Compared to the prior art, in which air from the bladder is passively vented to the ambient, the active transport from the bladder to a negative fluid reservoir, is much faster since the pressure difference between the fluid reservoir and the bladder is larger than the difference between the bladder and the ambient.

The fluid which is displaced from the positive reservoir to the negative reservoir in this way is then returned by the pump, such that a pressure differential between the two reservoirs is kept in place. The fluid may for instance be a gas or a gaseous mixture like air, or a liquid such as water.

The variable flow resistance may be arranged to completely open or close the connection between the fluid reservoirs and the bladder. This enables a discrete controlling of the connection.

It is also possible that the variable flow resistance only partially opens or closes the connection between the reservoirs and the bladder. The variable flow resistance controls the pressure in the bladder by regulating the flow resistance of fluid. Partly closing of the connection between the first fluid reservoir and the bladder, and opening the connection between the bladder and the second fluid reservoir, a flow from the bladder to the second reservoir is made easier, while flow from the first reservoir into the bladder is made harder. This results in a flow of fluid from the bladder, and thus in a pressure decrease in the bladder. The same holds the other way around.

The signal, typically the signal of the light detector, of the pressure cuff is representing the volume of blood inside the blood vessels of the body part. The more blood, the more light from the light source is scattered, which results in a lower signal (and vice versa). During every heartbeat, blood is forced through the blood vessels in the body part, causing the vessels to expand and allow more blood to flow through the vessels. This also causes a volume increase of the vessels, and thus a signal decrease.

The cuff pressure of the pressure cuff is controlled by the controller such that the signal, and thus the volume of blood inside the blood vessels, is kept constant. The pressure exerted on the blood vessel walls may be continuously counteracted by a pressure exerted by the pressure cuff, which may result in a constant diameter of the blood vessels and an unloading of the vessels. The counter pressure exerted by the pressure cuff then is a measure for the actual blood pressure inside the blood vessel, and allows for a continuous non-invasive blood pressure measurement.

The light source may comprise at least one LED, an infrared (IR) LED and/or any combinations hereof, and the light receiver may comprise at least one photodiode. The light source may also comprise a combination of LED's with mutually different wavelengths.

By using a device according to the invention, the amount of fluid being pumped around is reduced. Due to this reduced amount of air, a smaller sized pump compared to the prior art may be used, which in turn may use less energy. The proposed invention is thus more efficient compared to the prior art.

The device may comprise a pressure sensor, arranged between the variable flow resistance and the bladder. The pressure sensor senses the pressure in the bladder, which is a measure for the actual blood pressure inside the blood vessel. The sensor is in contact with the controller.

The pressure generator may be arranged to create a pressure difference between the first and second fluid reservoir of at least 150 mmHg, in particular at least 200 mmHg. The first fluid reservoir may for instance be around 100 mmHg above mean arterial pressure, and the second fluid reservoir may for instance be around 100 mmHg below mean arterial pressure. This way, the pressure generator may be able to counter various arterial pressures in patients, and react relatively quickly to pressure changes.

The pressure generator may also be arranged to create a pressure difference between the first and second fluid reservoir in dependence of the measured systolic and diastolic blood pressures, wherein the pressure difference for instance is created between a predetermined pressure above systolic pressure, and a predetermined pressure below diastolic pressure. The predetermined pressures could for instance be dependent on the systolic pressure and/or diastolic pressure measured, as well as to the measured heart rate.

The pressure generator may be arranged to create a pressure difference between the first and second fluid reservoir dependent on the measured blood pressure. The pressure generator could for instance be a pump which sucks air from one reservoir, and blows air in the other reservoir. The difference between the pressure inside the bladder and the pressure in the fluid reservoirs determines the speed in which fluid is flowing in and out of the bladder. When the pressures inside the reservoirs are kept at a constant pressure relative to the bladder pressure, the fluid flow from and into the bladder can be kept constant as well, since the pressure difference between the reservoirs and the bladder is kept the same. This allows for a more predictable system, in which reaction speed is independent from the pressure differences in the body part measured. The reservoirs may also be kept at a pressure relative to the minimum or maximal blood pressure measured, or the mean blood pressure.

The first and/or second fluid reservoir may comprise a valve, to regulate supply of ambient air to or from the first and/or second fluid reservoir when the pressure inside the reservoir reaches a predetermined pressure. When for instance the pressure inside the first fluid reservoir exceeds a threshold value, the valve of the first reservoir opens to expel excess pressure to the ambient. When for instance the pressure inside the second fluid reservoir drops below a threshold value, the valve of the second reservoir opens, to allow air from the ambient to flow into the second reservoir. This way build-up of undesired pressures inside the reservoirs can be avoided. The first and/or second fluid reservoir may also comprise a separate pump or pressure generating means, for adjusting the pressure inside the fluid reservoir independent on other fluid flows.

Advantageously, the pressure cuff and the variable flow resistance are arranged in a mobile device, such as a wearable wrist-unit. The device according to the invention is typically used to determine blood pressure inside the arteries of the finger of a patient, for instance in the index or middle finger. By locating the mobile device relatively close to the fingers, the unit can be worn comfortably. The mobile device could also be placed close to a subject, instead of on the subject.

The first and second fluid reservoirs may be arranged in the mobile device, such as the wearable wrist-unit. Because the volume of fluid inside the device according to the invention may be reduced to the substantially closed nature of the device, the reservoirs may be relatively small. This reduces the volume and weight the reservoirs require, and allows the reservoirs to be placed in the wearable wrist-unit, without a large burden for the wearer of the unit. It is also possible to arrange the pressure generator in the mobile device.

The device according to the invention may further comprise a monitor, for displaying the determined blood pressure. The first and/or second fluid reservoir may be arranged in the monitor. By locating the reservoirs in the monitor, instead of in the wrist unit, the weight of the wrist-unit can be reduced, which reduces the burden on the wearer of the wrist-unit. It is also possible to arrange the pressure generator in the monitor.

### BRIEF DESCRIPTION OF THE DRAWINGS

The invention will be explained by means of the non-limiting working examples depicted in the following figures. Specifically:

FIG. 1 schematically shows a device according to the invention in a block diagram;

FIG. 2 schematically shows a pressure cuff according to the invention; and

FIG. 3 schematically shows a device according to the invention

### DETAILED DESCRIPTION

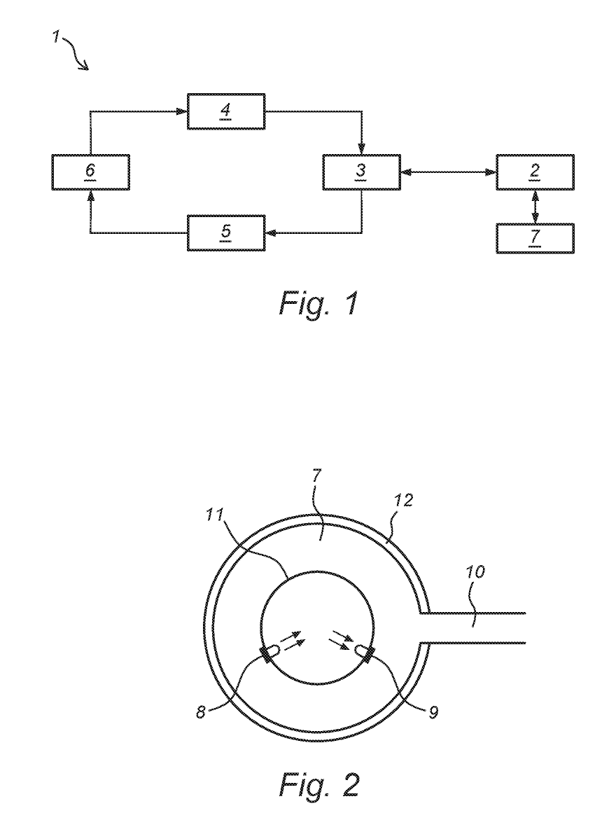

FIG. 1 schematically shows a device 1 for a non-invasive pressure blood pressure measurement, comprising a pressure cuff 2, for placement around a body part such as a finger. The cuff 2 is connected to a variable flow resistance 3, which controls the flow of fluid between the cuff 2, a first fluid reservoir 4 and a second fluid reservoir 5. Between the first 4 and second fluid reservoir 5 a pressure differential is created by a pressure generator 6. The cuff 2 comprises an inflatable bladder 7.

The variable flow resistance 3 is for instance formed by a valve or flapper 3. When the flow resistance 3 closes the fluid connection between the bladder 7 of the cuff 2 and the second fluid reservoir 5 completely, the bladder 7 is connected only to the first fluid reservoir 4 and vice versa. When the first fluid reservoir 4 is at a relatively high pressure, this causes a fluid flow from the first reservoir 4 to the bladder 7, which inflates the bladder.

In practise, the variable flow resistance 3 may only partially close off the connection between the bladder 7 and the fluid reservoirs 4, 5. Partial closing off will result in an increases resistance in flow from one of the reservoirs 4, 5 and a decreased resistance in flow from the other reservoir 5, 4. Not closing off the connection(s) completely prevents the unwanted pressure build-up inside the reservoirs 4, 5, as fluid can flow between them.

FIG. 2 schematically shows a pressure cuff 2, comprising an inflatable bladder 7, for wrapping around a body part such as a finger, a light source 8 and a light detector 9. The bladder 7 can be supplied with fluid through a line 10. By increasing the pressure in the bladder 7, the bladder walls 11 expand, which in turn exerts pressure on the (not shown) body part in the bladder 7. On the outside, the bladder 7 is surrounded by a rigid or semi-rigid housing 12, such that the bladder 7 expands substantially inward when inflated.

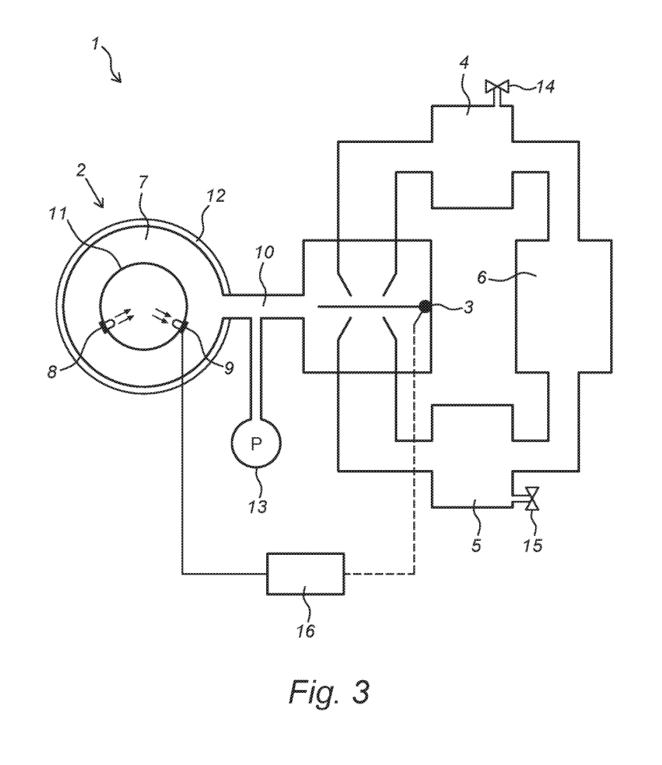

FIG. 3 schematically shows a device 1 according to the invention, comprising the pressure cuff 2 of FIG. 2, connected to a variable flow resistance 3, which controls the flow of fluid between the cuff 2, a first fluid reservoir 4 and a second fluid reservoir 5. Between the first 4 and second fluid reservoir 5 a pressure differential is created by a pressure generator 6. The cuff 2 comprises an inflatable bladder 7.

The variable flow resistance 3 is for instance formed by a valve or flapper 3. When the flow resistance 3 closes of the fluid connection between the bladder 7 of the cuff 2 and the second fluid reservoir 5 completely, the bladder 7 is connected only to the first fluid reservoir 4 and vice versa. When the first fluid reservoir 4 is at a relatively high pressure, this causes a fluid flow from the first reservoir 4 to the bladder 7, which inflates the bladder.

FIG. 3 further shows a pressure sensor 13, arranged between the variable flow resistance 3 and the bladder 7, for determining the pressure in the bladder 7. The first fluid reservoir 4 is provided with a first valve 14, and the second fluid reservoir 5 is provided with a second valve 15, to limit the pressure build-up inside the reservoirs. FIG. 3 further schematically shows a controller 16, which can control the flow resistance 3 based on the signal of the light detector 9. The controller 16 may also be connected to other elements of the system, to control these elements based on the signal of the light detector 9, or any other measuring system, as well.

It will be apparent that the invention is not limited to the exemplary embodiments shown and described here, but that within the scope of the appended claims numerous variants are possible which will be self-evident to the skilled person in this field.

1. A device for a non-invasive pressure blood pressure measurement, comprising:
a) a pressure cuff, for placement around a body part such as a finger; which cuff comprises:
a. a bladder, for applying a pressure to the body part;
b. a volume determining unit for providing a signal based on the volume of blood flowing through the body part, for example comprising:
i. a light source, for sending light through the body part; and
ii. a light detector, for detecting the light passed through the body part and for providing the signal in dependence of the amount of detected light;
b) a first fluid reservoir, in fluid connection with the bladder, to supply fluid to the bladder;
c) a second fluid reservoir, in fluid connection with the bladder, to receive fluid from the bladder;
d) a pressure generator, for generating a differential pressure between the first and second fluid reservoir;
e) a variable flow resistance, located between the fluid reservoirs and the bladder, for varying the flow between the reservoirs and the bladder; and
f) a controller, arranged to control the variable flow resistance based on the signal.

2. A device according to claim 1, further comprising a pressure sensor, arranged between the variable flow resistance and the bladder, for determining the pressure in the bladder.

3. A device according to claim 1, wherein the pressure generator is arranged to create a pressure difference between the first and second fluid reservoir of at least 150 mmHg, in particular at least 200 mmHg, or a pressure difference in dependence of a measured systolic and diastolic blood pressure.

4. A device according to claim 1, wherein the pressure generator is arranged to create a pressure difference between the first and second fluid reservoir dependent on the measured blood pressure

5. A device according claim 1, wherein the first and/or second fluid reservoir comprises a valve, to regulate supply of ambient air to or from the first and/or second fluid reservoir when the pressure inside the reservoir reaches a predetermined pressure.

6. A device according to claim 1, wherein the first and/or second fluid reservoir comprises a pump, for adjusting the pressure inside the fluid reservoir

7. A device according to claim 1, wherein the pressure cuff and the variable flow resistance are arranged in a wearable wrist-unit.

8. A device according to claim 7, wherein the first and second fluid reservoirs are arranged in the wearable wrist-unit.

9. A device according to claim 1, further comprising a monitor, for displaying the determined blood pressure

10. A device according to claim 9, wherein the first and/or second fluid reservoir are arranged in the monitor.
